(19) United States
(12) Patent Application Publication
(10) Pub. No.: US 2008/0294015 A1
(43) Pub. Date: Nov. 27, 2008

(54) Administration system

(75) Inventor: Katsuhisa Tsuji, Hoi-gun (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 12/153,633

(22) Filed: May 22, 2008

(30) Foreign Application Priority Data
May 25, 2007 (JP) 2007-139188

(51) Int. Cl. A61B 5/00

(52) U.S. Cl. 600/300

(57) ABSTRACT

An administration system proposes necessary exercise content in accordance with dining selections and a physical condition of a user. A portable terminal obtains and stores physical condition information and information on calories ingested upon dining. The portable terminal selects exercise content candidates based on the calorie ingestion information and the physical condition information and displays the selected exercise content candidates. A user can be informed before the start of exercise of necessary exercise contents corresponding to selected dining in consideration of physical condition.

### CROSS REFERENCE TO RELATED APPLICATIONS

The present invention is based on and claims priority to Japanese Patent Application No. 2007-139188, filed May 25, 2007 the entire contents of which are incorporated herein by reference.

### BACKGROUND OF THE INVENTION

1. Field of the Invention

The present invention relates to an administration system capable of administration of energy amounts ingested by dining.

2. Description of the Related Art

In accordance with rising health consciousness in recent years, various devices for administration of energy amounts, also referred to as “calories,” ingested during dining have been proposed. For example, a totalizing device described in Japanese Published Unexamined Patent Application No. 2003-61940 has a function of inputting ingested calories by wireless communication from a register at a restaurant or food shop, a function of inputting exercise intensity by wireless communication from a fitness machine at an athletic gym and calculating calorie expenditure, a function of inputting calories expended through singing from a karaoke console at a karaoke facility, and a function of obtaining total calorie expenditure in consideration of a basal metabolic rate. The total calorie expenditure, with calorie ingestion, is displayed on a liquid crystal panel.

However, the above-described totalizing device described in Japanese Published Unexamined Patent Application No. 2003-61940 merely displays the calorie ingestion and calorie expenditure in an ex-post manner. A user of the device cannot be previously informed of an exercise content the type and amount of exercise appropriate to the dining. The user may predict an exercise content from the displayed calorie ingestion. However, it is often difficult for the user to determine an appropriate exercise content. It is necessary to determine an exercise content in consideration of the physical condition of the user, however, the user may make a determination based on subjective factors and may be greatly influenced by factors such as exercise motivation leading to the entry of inaccurate information. Accordingly, the accuracy of the resulting determination may be low.

### SUMMARY OF THE INVENTION

The present invention has been made in consideration of the above problems, and provides an administration system capable of proposal of necessary exercise contents in accordance with dining in consideration of a physical condition of the user.

According to one aspect of the present invention, the foregoing object is attained by providing an administration system comprising first acquisition means for acquiring calorie information from a food provision facility terminal placed in a restaurant; second acquisition means for acquiring physical condition information as information on a physical condition of the user, necessary exercise content selection means for selecting a necessary exercise content based on the calorie information acquired by the first acquisition means while considering the physical condition information acquired by the second acquisition means, and output means for outputting the exercise content selected by the necessary exercise content selection means. Note that the phrase “while considering the physical condition information” means that a determination is made as to whether the user is in sufficiently good physical condition to withstand the exercise, and the degree of exercise load or the like appropriate to the physical condition of the user. Further, the term “exercise content” includes the type and amount of exercise.

According to the exemplary administration system, the user can be informed, for example, of an exercise content corresponding to a particular dining selection or several dining selections in consideration of the physical condition of the user, before exercise is begun.

Further, one user of the administration system may have a meal with another person. In such case, one conceivable problem is that the first acquisition means acquires calorie information from the food provision facility terminal other than that of the dining choice associated with the user. In such a situation, the necessary exercise content selection means cannot accurately select an exercise content. Accordingly, the first acquisition means should acquire a menu and the calorie information for each food and drink item on the menu including dishes, drinks, and the like, from the food provision facility terminal. The necessary exercise content selection means selects the exercise content based on the calorie information corresponding to a food selected by selection means from the menu. Note that operation buttons, a touch panel, a touch pen and the like may be used as the selection means.

According to the above-described administration system, even when the user has a meal with other persons, the calories ingested by the user can be accurately calculated and as a result, an exercise content can be accurately selected. Further, the physical condition information acquired by the second acquisition means is at least one of a body temperature, a heart rate, a respiration rate, blood pressure, a perspiration amount, a body weight, a basal metabolic rate, a muscle volume and a body fat percentage. Since the physical condition information is acquired as described above, an exercise content in consideration of the physical condition of the user can be easily selected.

The output means is a display means having an image display function. As the output means is capable of image display, the user can directly be informed of the exercise content simply with the administration system. The output means outputs reservation information based on the exercise content to a sport facility reservation management device. Reservations for the sport facility including the sporting equipment to be used can be made in accordance with the exercise content selected by the necessary exercise content selection means.

Among the constituent elements of the administration system, the first acquisition means is incorporated in a portable terminal, and the second acquisition means, the necessary exercise content selection means and the output means are mounted on a vehicle. Note that even when the system constituent elements are provided in separate bodies such as in the portable terminal and in the vehicle, information can be transmitted among the respective means to realize the functions of the respective means.

In such a case, when the user drives to a restaurant or the like, only the portable terminal is carried from the vehicle. The user can thereby easily obtain calorie information from a food provision facility terminal. That is, the system provides excellent usability. Further, among the constituent elements of the administration system, the first acquisition means, the necessary exercise content selection means and the output means are incorporated in a portable terminal, and the second acquisition means is mounted on a vehicle. Even in this case, when the user drives to a restaurant or the like, he/she carries only the portable terminal from the vehicle, thereby can easily obtain calorie information from a food provision facility terminal. That is, the system provides excellent usability.

Note that the second acquisition means acquires the physical condition information from a driver monitor sensor provided in the vehicle. The driver monitor sensor may be a body temperature sensor, a heart rate sensor, a respiration sensor and an electric conductivity sensor provided in a steering wheel, a weight sensor provided in a seat, a driver monitor camera provided in an instrument panel, and the like. In such a case, it is unnecessary to provide a sensor specialized for the administration system contributing to cost reduction associated with the manufacture of the administration system.

### BRIEF DESCRIPTION OF THE DRAWINGS

The above and other object, features and advantages of the present invention will become more apparent from the following detailed description when taken in conjunction with the accompanying drawings wherein:

FIG. 1 is a block diagram illustrating an exemplary administration system according to a first embodiment;

FIG. 2 is a flowchart illustrating exemplary calorie intake processing;

FIG. 3 is a flowchart illustrating exemplary physical condition information acquisition processing;

FIG. 4 is a flowchart illustrating exemplary exercise content determination processing;

FIG. 5 is a flowchart illustrating exemplary notification processing;

FIG. 6 is a block diagram illustrating an exemplary administration system according to a second embodiment;

FIG. 7 is a flowchart illustrating exemplary calorie intake processing; and

FIG. 8 is a flowchart illustrating exemplary exercise content determination processing.

### DETAILED DESCRIPTION OF THE EXEMPLARY EMBODIMENTS

Exemplary embodiments will now be described in detail in accordance with the accompanying drawings. Note that the embodiments are not limited to the following embodiments, and various changes and modifications can be made within the technical scope of the invention.

### First Embodiment

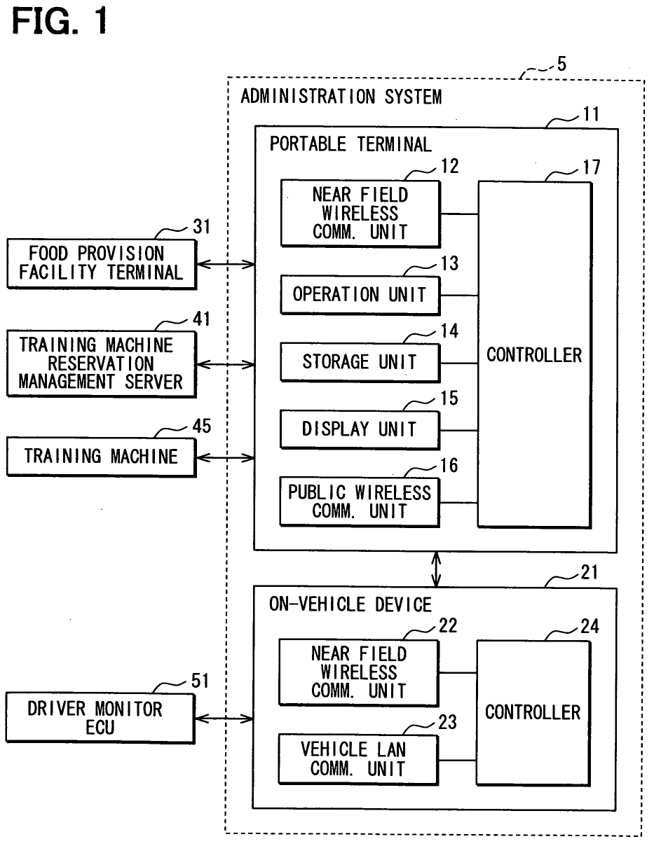

In a first embodiment, various processing is performed mainly using a portable terminal. FIG. 1 shows an administration system 5 according to a first embodiment. The administration system 5 includes a portable terminal 11 and an on-vehicle device 21. The portable terminal 11 is capable of communicating with a terminal 31, a server 41 and a training machine 45. The on-vehicle device 21 is capable of communicating with a driver monitor electronic control unit (ECU) 51.

The portable terminal 11 has a near field wireless communication unit 12, an operation unit 13, a storage unit 14, a display unit 15, a public wireless communication unit 16 and a controller 17. The near field wireless communication unit 12 has a function of communicating with the terminal 31, the training machine 45 and the on-vehicle device 21, by near field wireless communication such as wireless LAN communication, short range communication such as can be achieved using a BLUETOOTH® brand short range communication enabled device, or other close proximity-based communication. The operation unit 13, includes operation buttons, a touch panel and the like, and has a function of receiving operational input from a user. The storage unit 14, includes a nonvolatile storage medium such as a flash memory that has a function of holding various data. The display unit 15 includes an image display panel such as a liquid crystal panel, an organic EL panel, or the like and has a function of displaying various characters, images, figures and the like.

The public wireless communication unit 16 has a function of communicating with the server 41 by public wireless communication such as cellular phone communication or wireless LAN communication, or the like including for example, packet communication.

The controller 17, having a well-known CPU, a ROM, a RAM, an I/O and the like, has a function of controlling the above units based on a program stored in the ROM or the storage unit 14 and performing various processing.

The on-vehicle device 21 has a near field wireless communication unit 22, a vehicle LAN communication unit 23 and a controller 24. The near field wireless communication unit 22 has a function of communicating with the portable terminal 11 by near field wireless communication such as wireless LAN communication, short range BLUETOOTH® brand communication as described above, or other close proximity-based communication or communication protocol. The vehicle LAN communication unit 23, connected to a vehicle LAN (not shown), has a function of communicating with the driver monitor ECU 51 via the vehicle LAN. The controller 24, having a well-known CPU, a ROM, a RAM, an I/O and the like, controls the above described units based on a program stored in the ROM and performs various processing.

The food provision facility terminal 31 (hereinafter “terminal 31”) has a function associated with, for example, a register placed in a restaurant, a convenience store, or the like. Note that the terminal 31 holds calorie information on foods and drinks offered on the restaurant menu, the calorie information being entered, for example, during a registration process. The terminal 31 transmits calorie information regarding the registered foods upon check-out to the portable terminal 11 by near field wireless communication as previously described.

The training reservation management server 41 (hereinafter “server 41”), which is managed by a sports club operating company or the like, manages reservations for training machines, group or individual exercise sessions, group or individual sports or sports training sessions, massage service, esthetique service and the like. In the first embodiment, the server 41 is capable of communicating with the portable terminal 11 via public wireless communication as previously described.

The training machine 45 can be an exercise machine such as a running machine, a cycle machine or a climber machine installed in a sports club. Note that the training machine 45 performs communication with the portable terminal 11 via near field wireless communication, as described above, and transmits calorie information on calories expended through use of the training machine 45 to the portable terminal 11.

The driver monitor ECU 51 has a function of communicating with a driver monitor sensor so as to estimate physical condition information of the vehicle driver. The driver monitor sensor can include, for example, a body temperature sensor, a heart rate sensor, a respiration sensor and an electric conductivity sensor provided in a steering wheel, a weight sensor provided in a seat, a driver monitor camera provided in an instrument panel, or the like. The physical condition information can include, for example, a body temperature, a heart rate, a respiration rate, blood pressure, a perspiration amount, a body weight, a basal metabolic rate, a muscle volume, a body fat percentage, or the like.

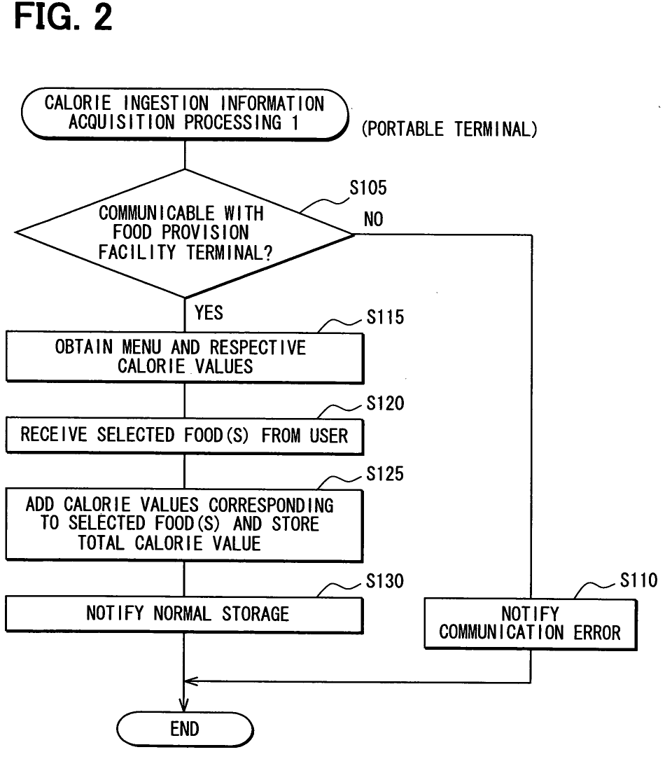

The operation of the administration system 5 can be described as follows. A calorie intake processing 1, which is associated with calorie ingestion information acquisition processing performed by the controller 17, will be described using the flowchart of FIG. 2. Note that the calorie intake processing 1 is started when a start command is received from the user via the operation unit 13.

When the calorie intake processing 1 is started, the controller 17 first determines whether communication with the terminal 31 can be performed via the near field wireless communication unit 12 at S105. When the controller 17 determines that communication with the terminal 31 can be performed, corresponding to YES at S105, the controller 17 proceeds to S115. When the controller 17 determines that communication with the terminal 31 cannot be performed, corresponding to NO at S105, processing proceeds to S110.

At S110, the controller 17 causes the display unit 15 to display a message indicating that communication is impossible. Note that various reasons for communication failure are conceivable. For example, the terminal 31 is not in an active communication status, or the terminal 31 is located beyond a communication distance from the portable terminal 11. Thereafter, the controller 17 terminates the processing such as calorie intake processing 1.

At S115, when communication is possible, the controller 17 obtains a menu including dish names or the like, and respective calorie values. More particularly, food information, such as “Ramen (Chinese noodle soup)” along with a calorie value “700 kcal”, is obtained for each food. The food information is displayed and the food selection of the user is input at S120.

More particularly, the controller 17 causes the display unit 15 to display the menu including dish names and calorie values in a list form such that the user can selects the food actually consumed via the operation unit 13. Note that the menu information may be arranged for acquisition of more accurate information such as that the food was only partially eaten or that a buffet-style restaurant was used and a particular number of portions were consumed. Corrections on calorie values can also be received from the user upon reception of food selection. Further, the input display may be arranged such that the user can manually input the ingested calorie values, for example, when dining at home.

Next, the controller 17 adds calorie values corresponding to the selected food items at S120, and stores the total calorie value as calorie ingestion information into the storage unit 14 at S125. For example, the total calorie value is obtained through simple addition such as 700 kcal (calorie value of ramen)+350 kcal (calorie value of steam-baked meat pie)=1050 kcal (total calorie value), with the result being stored into the storage unit 14. Thereafter, the controller 17 causes the display unit 15 to display a message indicating that the total calorie value has been normally stored at S130. Then the controller 17 terminates the processing such as the calorie intake processing 1.

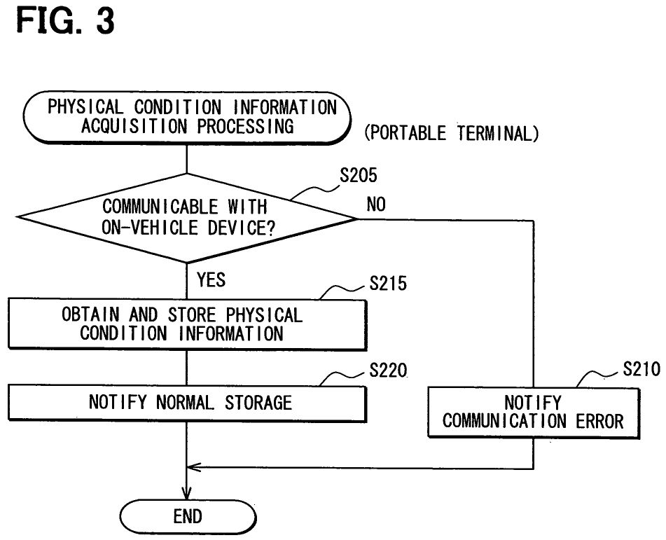

Next, physical condition processing, which includes physical condition information acquisition processing, performed by the controller 17 will be described with reference to the flowchart of FIG. 3. Note that the physical condition processing is started when a start command is received from the user via the operation unit 13. Further, it is presumed that the physical condition processing is performed when the user is seated on a seat of the vehicle.

When the physical condition processing is started, the controller 17 first determines whether communication with the on-vehicle device 21 can be performed via the near field wireless communication unit 12 at S205. When the controller 17 determines that communication with the on-vehicle device 21 can be performed, corresponding to YES at S205, the controller 17 proceeds to S215. When the controller 17 determines that communication with the on-vehicle device 21 cannot be performed, corresponding to NO at S205, processing proceeds to S210.

At S210, the controller 17 causes the display unit 15 to display a message indicating that communication is impossible. Note that various reasons for communication failure are conceivable. For example, the on-vehicle device 21 is not in an active communication status, or the on-vehicle device 21 is located beyond a communication distance from the portable terminal 11. Thereafter, the controller 17 terminates the physical condition processing. At S215, when communication with the on-vehicle device 21 can be performed, the controller 17 obtains the physical condition information from the driver monitor ECU 51 via the on-vehicle device 21 and stores the information into the storage unit 14. The controller 17 causes the display unit 15 to display a message indicating that the physical condition information has been normally stored at S220. The controller 17 then terminates the physical condition processing.

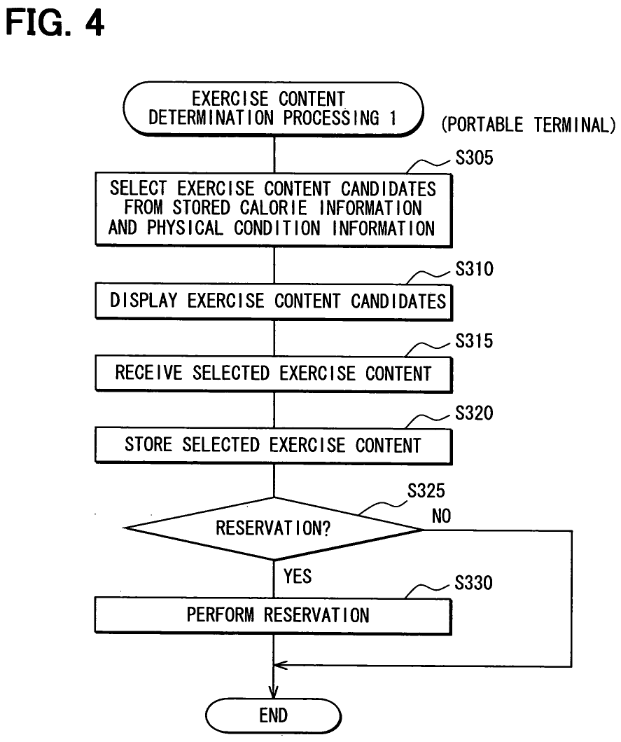

Next, exercise content determination processing 10 performed by the controller 17 will be described with reference to the flowchart of FIG. 4. Note that the exercise content determination processing 10 is started when a start command is received from the user via the operation unit 13, on the condition that the calorie ingestion information and the physical condition information are stored in the storage unit 14.

When the controller 17 starts the exercise content determination processing 10, exercise content candidates are first selected from the calorie ingestion information and the physical condition information stored in the storage unit 14 at S305. That is, the controller 17 determines whether the user is in good physical condition to withstand the exercise, and determines the degree of exercise load and the like appropriate to the physical condition of the user. Then the controller 17 selects combinations of exercise types and exercise amounts exercise as exercise content candidates based on the calorie ingestion information.

Next, the controller 17 causes the display unit 15 to display the exercise content candidates selected at S305 at S310. The controller 17 then receives a selected exercise content candidate from the user via the operation unit 13 at S315, and stores the selected exercise content candidate into the storage unit 14 at S320. Next, the controller 17 determines whether a reservation for a training machine, exercise session, massage service, esthetique service or the like is to be made based on the selected exercise content at S325. The determination is made by, for example, displaying a message asking the user whether a reservation is to be made on the display unit 15, and determining whether an operation to instruct the reservation has been performed on the operation unit 13 by the user. As a result, when the controller 17 determines that a reservation for a training machine or the like is not to be made, corresponding to NO at S325, the controller 17 terminates the processing, such as the exercise content determination processing 10. On the other hand, when the controller 17 determines a reservation for a training machine or the like is to be made, corresponding to YES at S325, the controller 17 proceeds to S330.

At S330, a reservation for a training machine or the like is made. More particularly, the controller 17 transmits reservation information to the server 41 via the public wireless communication unit 16, to make a request for reservation for a training machine, exercise session, massage service, esthetique service or the like. Note that the reservation information includes date and time of visit, a reservation object, user identification information and the like. Thereafter, the controller 17 terminates the processing such as the exercise content determination processing 10.

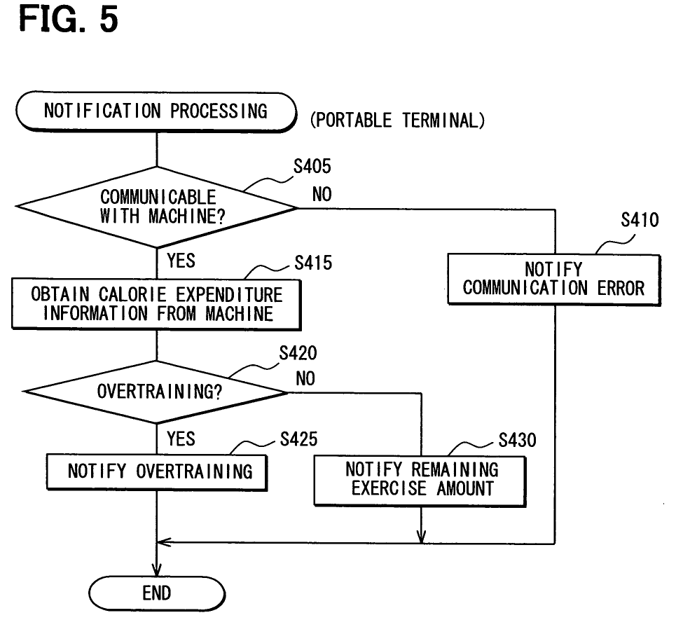

Next, notification processing performed by the controller 17 will be described with reference to the flowchart of FIG. 5. Note that the notification processing, which may be performed periodically, is started when a start command is received from the user via the operation unit 13 provided that the exercise content is stored in the storage unit 14. Further, it is basically presumed that the notification processing is performed by the user during or after use of the training machine 45.

When the controller 17 starts the notification processing, the controller 17 first determines whether communication with the training machine 45 can be performed at S405. When the controller 17 determines that communication with the training machine 45 can be performed, corresponding to YES at S405, the controller 17 proceeds to S415. When the controller 17 determines that communication with the training machine 45 cannot be performed, corresponding to NO at S405, processing proceeds to S410.

At S410, when communication cannot be performed, the controller 17 causes the display unit 15 to display a message indicating that communication is impossible. Note that various reasons for communication failure are conceivable. For example, the training machine 45 is not in an active communication status, or the training machine 45 is located beyond a communication distance from the portable terminal 11. Thereafter, the controller 17 terminates the notification processing. At S415 when communication can be performed, the controller 17 obtains calorie expenditure information from the training machine 45 via the near field wireless communication unit 12 as described above. The calorie expenditure information refers to calories expended through exercise using the training machine 45. Note that, in place of the above calories, a physical quantity used for calculation of the calories such as a gait distance or the like, is obtained from the training machine 45, and the calorie expenditure is calculated in consideration of, for example, the body weight of the user, or the like, in the portable terminal 11.

Next, the controller 17 determines whether the user is in an overtraining status at S420. That is, the controller 17 compares the calorie ingestion information stored in the storage unit 14 with the calorie expenditure information obtained at S415, and determines whether the calorie expenditure information is higher than the calorie ingestion information. Note that a more detailed determination can be made in consideration of, for example, an elapsed time from dining or the basal metabolic rate of the user in place of such simple comparison between calorie intake and expenditure.

At S420, when the controller 17 determines that the user is in an overtraining status, corresponding to YES at S420, the controller 17 proceeds to S425. When the controller 17 determines that the user is not in an overtraining status, corresponding to NO at S420, processing proceeds to S430.

At S425, when the user is in an overtraining status, the controller 17 causes the display unit 15 to display a message indicating overtraining, and terminates the notification processing. At S430 when the user is not in an overtraining status, the controller 17 causes the display unit 15 to display a remaining exercise amount, and terminates the notification processing.

The portable terminal 11 according to the above describe first embodiment obtains and stores the physical condition information in addition to the calorie ingestion information at S215. The portable terminal 11 then selects exercise content candidates based on the calorie ingestion information and the physical condition information at S305, and displays the selected exercise content candidates at S310. Accordingly, the user can be informed of exercise contents which have been selected in consideration of his/her physical condition and become necessary in accordance with dining before start of exercise.

Further, the portable terminal 11 receives and stores a selected one of the displayed exercise content candidates from the user at S315 and S320, and makes a reservation for a training machine, exercise session, massage service, esthetique service or the like based on the exercise content selected by the user at S330. Accordingly, the user can make a reservation for a training machine or the like corresponding to the exercise content without making a reservation telephone call or attempting to exercise without a reservation.

Further, the portable terminal 11 performs communication with the training machine 45, and obtains information on calories expended through exercise using the training machine 45 at S415, and determines whether the calorie expenditure exceeds the calorie ingestion at S420. When the calorie expenditure exceeds the calorie ingestion, corresponding to YES at S420, the portable terminal 11 notifies the user of overtraining status at S425. The user can check the notification, thereby preventing overtraining.

Further, as the portable terminal 11 obtains and stores the physical condition information from the driver monitor ECU 51 mounted on the vehicle via the on-vehicle device 21 at S215, it is not necessary for the user to input the physical condition information via the operation unit 13. Further, it is not necessary to provide specialized sensors, and the like. Accordingly, the system provides excellent usability and low cost.

Next, it will be noted that a correspondence between the above described terms of the embodiment and the claim terms will be given. The near field wireless communication unit 12 can correspond to the first acquisition means, the on-vehicle device 21 can correspond to the second acquisition means, the controller 17 can correspond to the necessary exercise content selection means, the display unit 15 can correspond to the output means and the display means, and the operation unit 13 can correspond to the selection means.

### Second Embodiment

A second embodiment can be envisioned that is different from the first embodiment in that a part of the processing is performed by the on-vehicle device in place of the portable terminal.

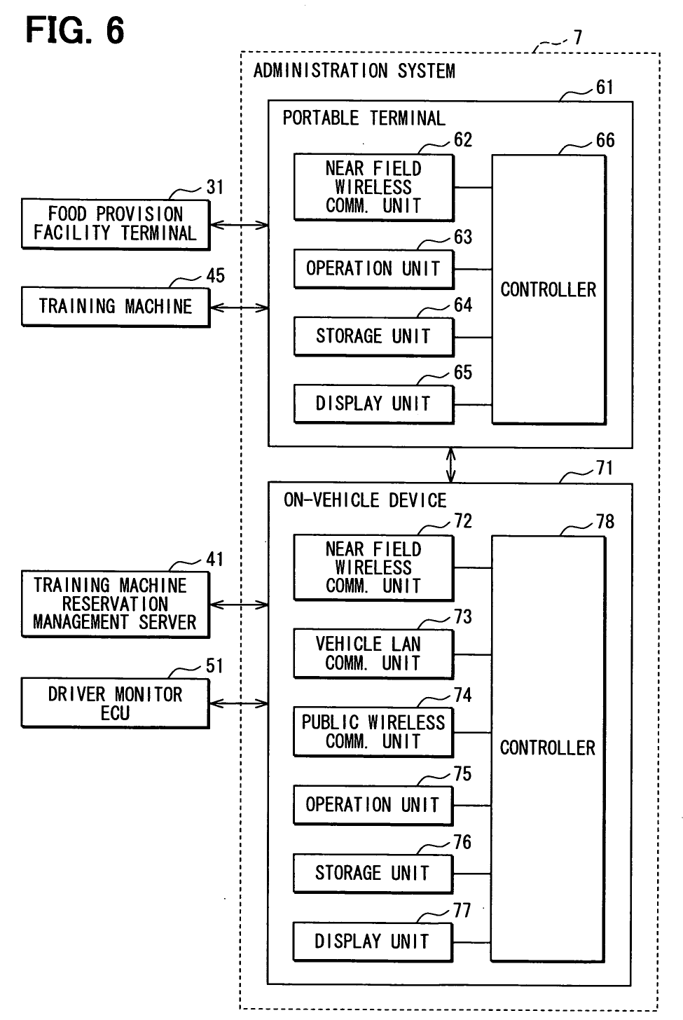

The block diagram of FIG. 6 shows an administration system 7 according to a second embodiment. The administration system 7 includes a portable terminal 61 and an on-vehicle device 71. The portable terminal 61 is capable of communicating with the terminal 31 and the training machine 45. The on-vehicle device 71 is capable of communicating with the server 41 and the driver monitor ECU 51. The system will be described in detail herein below, however, the constituent elements corresponding to those in the first embodiment will have the same reference numerals, and the explanations thereof will be omitted.

The portable terminal 61 has a near field wireless communication unit 62, an operation unit 63, a storage unit 64, a display unit 65 and a controller 66. The near field wireless communication unit 62 has a function of communicating with the portable terminal 61, the on-vehicle device 71, and the training machine 45 by near field wireless communication such as described herein above. The operation unit 63, having operation buttons, a touch panel and the like, has a function of receiving operation input from a user. The storage unit 64, having a nonvolatile storage medium such as a flash memory, has a function of holding various data.

The display unit 65, includes an image display panel such as a liquid crystal panel, an organic EL panel, or the like, and has a function of displaying various characters, images, figures and the like. The controller 66, includes a well-known CPU, a ROM, a RAM, an I/O and the like, and has a function of controlling the above units based on a program stored in the ROM or the storage unit 64 and performing various processing.

The on-vehicle device 71 has a near field wireless communication unit 72, a vehicle LAN communication unit 73, a public wireless communication unit 74, an operation unit 75, a storage unit 76, a display unit 77 and a controller 78. The near field wireless communication unit 72 has a function of communicating with the portable terminal 61 by near field wireless communication as previously described. The vehicle LAN communication unit 73, connected to a vehicle LAN (not shown), has a function of communicating with the driver monitor ECU 51 via the vehicle LAN. The public wireless communication unit 74 has a function of communicating with a training machine reservation server by public wireless communication as previously described.

The operation unit 75, includes operation buttons, a touch panel or the like, and has a function of receiving the operation input from a user. The storage unit 76, includes a nonvolatile storage medium such as a flash memory, and has a function of holding various data. The display unit 77, includes an image display panel such as a liquid crystal panel or an organic EL panel, and has a function of displaying various characters, figures and the like. The controller 78, includes a well-known CPU, a ROM, a RAM, an I/O and the like, has a function of controlling the above units based on a program stored in the ROM and performing various processing.

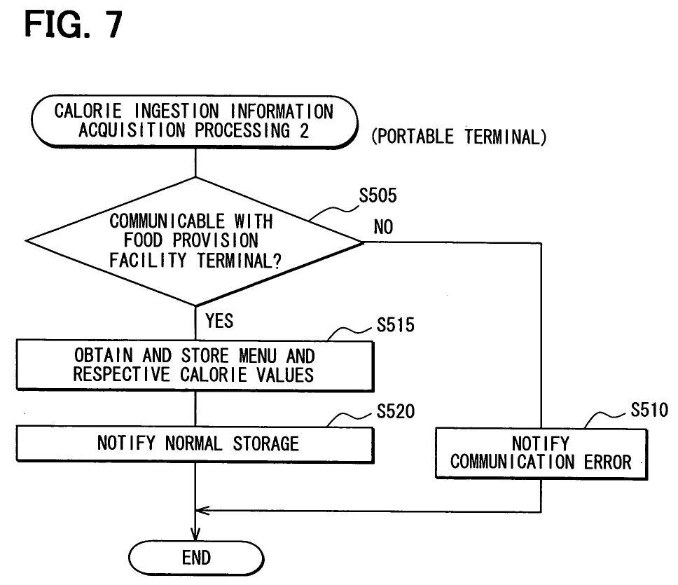

Next, the operation of the administration system 7 will be described. First, calorie intake processing 2, which includes calorie ingestion information acquisition processing performed by the controller 66, will be described using the flowchart of FIG. 7. Note that the calorie intake processing 2 is started when a start command is received from the user via the operation unit 63. When the controller 66 starts the calorie intake processing 2, it is first determined whether communication with the portable terminal 61 can be performed via the near field wireless communication unit 62 at S505. When the controller 66 determines that communication can be performed, corresponding to YES at S505, the controller 66 proceeds to S515. When the controller 66 determines that communication cannot be performed, corresponding to NO at S505, processing proceeds to S510.

At S510, when communication cannot be performed, the controller 66 causes the display unit 65 to display a message indicating that communication is impossible. The controller 66 then terminates the calorie intake processing 2. At S515, when communication can be performed, the controller 66 obtains a menu such as dish names or the like, and respective calorie values and stores them into the storage unit 64. More particularly, information, such as a food “Ramen” and a calorie value “700 kcal,” are obtained for each food. Thereafter, the controller 66 controls the display unit 65 to display a message indicating that the information has been normally stored at S520. Then the controller 66 terminates the calorie intake processing 2.

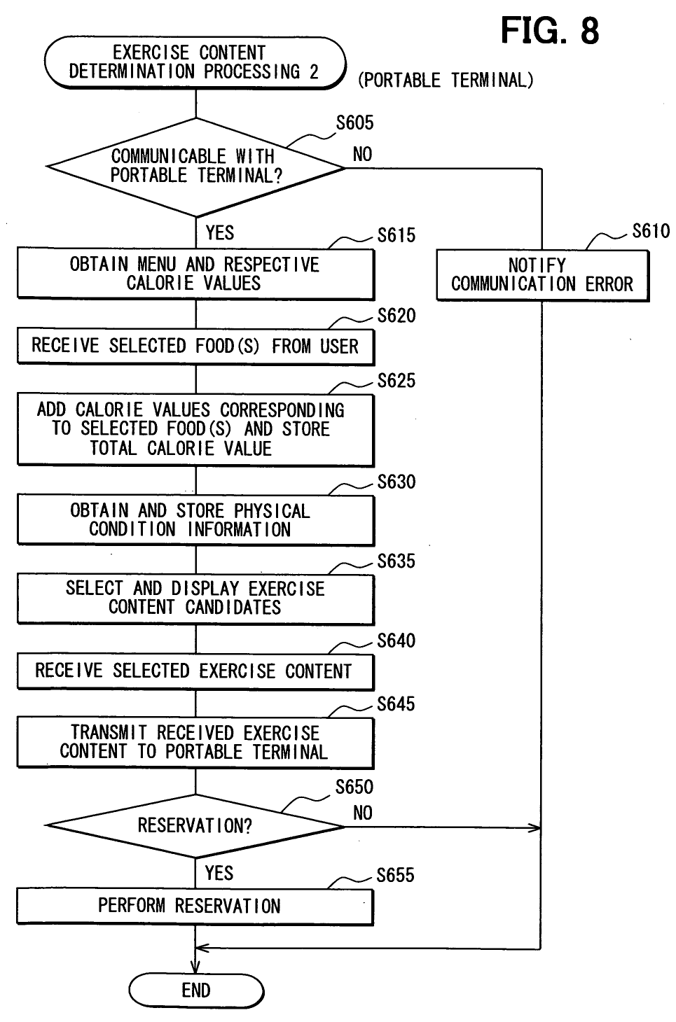

Next, exercise content determination processing 20 performed by the controller 78 will be described with reference to the flowchart of FIG. 8. Note that the exercise content determination processing 20 is started when a start command is received from the user via the operation unit 75. Further, it is presumed that the exercise content determination processing 20 is performed when the user is seated in the seat of the vehicle.

When the controller 78 starts the exercise content determination processing 20, it is first determined whether communication with the portable terminal 61 can be performed via the near field wireless communication unit 72 at S605. When the controller 78 determines that communication can be performed, corresponding to YES at S605, the controller 78 proceeds to S615. When the controller 78 determines that communication with the portable terminal 61 cannot be performed, corresponding to NO at S605, processing proceeds to S610.

At S610, when communication cannot be performed, the controller 78 causes the display unit 77 to display a message indicating that communication is impossible. Then the controller 78 terminates the exercise content determination processing 20. At S615, when communication can be performed, the controller 78 obtains a menu containing dish names or the like, and respective calorie values that are stored in the storage unit 64 from the portable terminal 61.

A food selection of the user is received at S620. That is, the controller 78 causes the display unit 77 to display the menu items such as dish names and the like in a list such that the user can select the food items actually consumed via the operation unit 75. The controller 78 adds calorie values corresponding to the selected items at S620, and stores the total calorie value as calorie ingestion information into the storage unit 76 at S625. The controller 78 obtains the physical condition information from the driver monitor ECU 51 and stores the information into the storage unit 76 at S630. The controller 78 then selects exercise content candidates from the calorie ingestion information and the physical condition information stored in the storage unit 76, and controls the display 77 to display the selected exercise content candidates at S635. That is, the controller 78 determines whether the user is in good physical condition to withstand the exercise, and determines the degree of exercise load and the like appropriate to the physical condition of the user based on the physical condition information. The controller 78 selects combinations of exercise types and exercise amounts as exercise content candidates based on the calorie ingestion information.

The controller 78 receives a selected exercise content from the user via the operation unit 75 at S640 and transmits the selected exercise content to the portable terminal 61 via the near field wireless communication unit 72 at S645. The controller 78 determines whether a reservation for a training machine, exercise session, massage service, esthetique service or the like is to be made based on the exercise content selected at S640 at S650. The determination is made by, for example, displaying a message asking the user whether a reservation is to be made on the display unit 77, and determining whether an operation to complete the reservation has been performed on the operation unit 75 by the user. As a result, when the controller 78 determines that a reservation for a training machine or the like is not to be made, corresponding to NO at S650, the controller 78 terminates the exercise content determination processing 20. When the controller 78 determines a reservation for a training machine or the like is to be made, corresponding to YES at S650, the controller 78 proceeds to S655.

At S655, a reservation for a training machine or the like is made. More particularly, the controller 78 transmits reservation information to the server 41 via the public wireless communication unit 74 in order to make a request for reservation for a training machine, exercise session, massage service, esthetique service or the like. Note that the reservation information includes date and time of visit, a reservation object, user specifying information and the like. Thereafter, the controller 78 terminates the exercise content determination processing 20.

The controller 66 performs notification processing in accordance with that performed according to the first embodiment. Therefore, the explanation of the processing will be omitted for simplicity.

The on-vehicle device 71 according to the second embodiment obtains and stores the physical condition information in addition to the calorie ingestion information at S615, S625 and S630. Then the on-vehicle device 71 selects exercise content candidates based on the calorie ingestion information and the physical condition information, and displays the selected exercise content candidates at S635. Accordingly, the user can be informed of exercise contents that have been selected in consideration of the physical condition of the user and that will become necessary in accordance with dining, before start of exercise.

Further, the on-vehicle device 71 receives a selected one of the displayed exercise content candidates from the user at S640, and makes a reservation for a training machine, exercise session, massage service, esthetique service or the like based on the exercise content selected by the user at S655. Accordingly, the user can make a reservation for a training machine or the like corresponding to the exercise content without making a reservation telephone call or the like. It should be noted that since the portable terminal 61 in the administration system 7 according to the second embodiment lacks a constituent element corresponding to the public wireless communication unit 16 in the first embodiment, the portable terminal 61 can be a lightweight unit with enhanced portability.

Next, exemplary correspondence between claim terms and terms used in the above description will be given. For example, the near field wireless communication unit 62 can correspond to the first acquisition means, the on-vehicle device 71 can correspond to the second acquisition means, the controller 78 can correspond to the necessary exercise content selection means, the display unit 77 can correspond to the output means and the display means, and the operation unit 75 can correspond to the selection means.

In the above embodiments, the portable terminal 11 and 61 obtain the calorie expenditure information from the training machine 45. However, it may be arranged such that the portable terminal 11 and 61 transmit the calorie ingestion information to the training machine 45, which can then determine whether the user is in an overtraining status from the calorie ingestion information and the calorie expenditure information, and notifies the result of determination.

The above arrangement has similar advantages to those of the above-described embodiments.

What is claimed is:

1. An administration system comprising:
first acquisition means for acquiring calorie information from a food provision facility terminal in a dining facility;
second acquisition means for acquiring physical condition information associated with a physical condition of a user;
necessary exercise content selection means for selecting a necessary exercise content based on the calorie information acquired by the first acquisition means while considering the physical condition information acquired by the second acquisition means; and
output means for outputting the exercise content selected by the necessary exercise content selection means.

2. The administration system according to claim 1, wherein the first acquisition means acquires a menu and the calorie information for each food of the menu from the food provision facility terminal, and
wherein the necessary exercise content selection means selects the exercise content based on the calorie information corresponding to a food selected by selection means from the menu.

3. The administration system according to claim 1, wherein the physical condition information acquired by the second acquisition means includes one of a body temperature, a heart rate, a respiration rate, blood pressure, a perspiration amount, a body weight, a basal metabolic rate, a muscle volume and a body fat percentage.

4. The administration system according to claim 1, wherein the output means includes a display means having an image display function.

5. The administration system according to claim 1, wherein the output means outputs reservation information based on the exercise content to an exercise facility reservation management device.

6. The administration system according to claim 1, wherein the first acquisition means is incorporated in a portable terminal, and
wherein the second acquisition means, the necessary exercise content selection means and the output means are mounted on a vehicle.

7. The administration system according to claim 1, wherein the first acquisition means, the necessary exercise content selection means and the output means are incorporated in a portable terminal, and
wherein the second acquisition means is mounted on a vehicle.

8. The administration system according to claim 6, wherein the second acquisition means acquires the physical condition information from a driver sensor provided in the vehicle.

9. The administration system according to claim 7, wherein the second acquisition means acquires the physical condition information from a driver sensor provided in the vehicle.

10. A vehicle mounted terminal for coupling to a system for administering health related information and activities of a user of a vehicle, the vehicle mounted terminal comprising:
a first interface capable of a near field communication for connecting, when within a predetermined near field range, with a portable terminal;
a second interface capable of a vehicle network communication for connecting with an electronic control unit (ECU), the ECU configured to monitor a physical condition of the user; and
a controller coupled to the first interface, the second interface, an input unit and a display unit,
wherein the controller is configured to:
receive a list of menu selections and corresponding calorie information from the portable terminal, when within the predetermined near field range, over the first interface, the controller configured to display the list of menu selections on the display unit and receive, from the input unit, which of the menu selections was consumed by the user;
receive the physical condition of the user from the ECU over the second interface; and
display a list of exercise content candidates on the display based on the calorie information associated with the consumed ones of the menu selections based and the physical condition of the driver.

11. The vehicle mounted terminal according to claim 10, further comprising a third interface capable of a public wireless network communication for connecting with a reservation server associated with a facility that offers at least some of the exercise content candidates, wherein the controller is further configured to receive which of the list of exercise content candidates is selected by the user and making a reservation associated with the selected one of the exercise content candidates with the reservation server over the third communication interface.

12. The vehicle mounted terminal according to claim 10, wherein the physical condition information includes at least one of a body temperature, a heart rate, a respiration rate, blood pressure, a perspiration amount, a body weight, a basal metabolic rate, a muscle volume and a body fat percentage.

13. A portable terminal for coupling to a system for administering health related information and activities of a user of a vehicle, the portable terminal comprising:
a first interface capable of a near field communication, the near field communication for connecting, when within a predetermined near field range, with a terminal, a server, a training machine and a vehicle mounted terminal;
a second interface capable of a public wireless network communication, the public wireless network communication for connecting with the server when not within the predetermined near field range thereof; and
a controller coupled to the first interface, the second interface a display and an input unit,
wherein:
the terminal includes a food provision facility terminal in a dining facility and, when within the predetermined near field range, the terminal transmits a list of menu selections and corresponding calorie information over the first interface, the controller configured to display the list of menu selections and receive an input associated with which of the menu selections was consumed by the user;
the vehicle mounted unit, when within the predetermined near field range, transmits a physical condition of the user over the first interface; and
the controller is configured to display a list of exercise content candidates based on the calorie information associated with the consumed ones of the menu selections based and the physical condition of the driver.

14. The portable terminal according to claim 13, wherein the controller is further configured to receive which of the list of exercise content candidates is selected by the user and making a reservation associated with the selected one of the exercise content candidates with the server over one of the first and the second interface.

15. The portable terminal according to claim 13, wherein the physical condition information includes at least one of a body temperature, a heart rate, a respiration rate, blood pressure, a perspiration amount, a body weight, a basal metabolic rate, a muscle volume and a body fat percentage.
